(19) United States
(12) Patent Application Publication
(10) Pub. No.: US 2005/0090651 A1
(43) Pub. Date: Apr. 28, 2005

(54) Polymeric compounds

(75) Inventors: Richard Anthony Godwin Smith, Essex (GB); Jason Richard Betley, Essex (GB); Dirk Esser, Essex (GB)

Correspondence Address: HELLER EHRMAN WHITE & MCAULIFFE LLP, 1717 RHODE ISLAND AVE, NQ, WASHINGTON, DC, 20036-3001, US

(73) Assignee: AdProTech Limited Cesterford Research Park, Little Chesterford Saffr, Essex (GB)

(21) Appl. No.: 10/491,216

(22) PCT Filed: Sep. 26, 2002
(86) PCT No.: PCT/GB02/04352

(30) Foreign Application Priority Data
Sep. 27, 2001 (GB) 0123262.8`

(51) Int. Cl. C07K 14/00; C07D 279/12

(52) U.S. Cl. 530/408; 544/59

(57) ABSTRACT

The invention provides a process for forming a compound having the formula (I): from chemical entities native [A], native [B] and native [C], where native [A] has a thioester group, native [B] had a 1-amino-2-thiol group with an unoxidised sulfhydryl side chain, and native [C] had a thiol reactive function (TRF) group, wherein chemical entities [A], [B] and [C] are covalently linked by linker group L, comprising the steps of: (i) admixing native [A] and native [B] in a reaction solution; (ii) condensing the unoxidised sulfydryl side chain of native [B] with the thioester group of native [A] for producing a first intermediate compound wherein [A] and [B] are linked with a β-aminothioester bond; (iii) rearranging the β-aminothioester bond for producing a second intermediate compound wherein [A] and [B] are linked with an amide bond having attached thereto a free thiol group; (iv) admixing native [C] with the second intermediate compound in a reaction solution; and (v) reacting the thiol reactive function (TRF) group of native [C] with the free thiol group of the second intermediate compound for producing a compound having the formula (I).

This invention relates to assembly of polymeric compounds using native chemical ligation as a method step. In particular, it relates to polypeptide assemblies, their use in therapy, and methods and intermediates for their production.

### BACKGROUND TO THE FIELD OF INVENTION

Combination therapy is a widely used approach to many diseases in the clinic It is found frequently that the effects of administering two or more drugs for the same indication of only modest efficacy may result in a clinical effect significantly greater than the sum of the parts. Such therapies are used widely in diseases such as cancer and AIDS.

Ligand directed therapy is another recent development that has received wide attention. The therapy relies on a pharmacophore (drug) being covalently or non-covalently attached to a ligand specific for a certain cell surface marker or tissue. The ligand directs the drug to the surface of the cell where it may exhibit its action at an increased local concentration. This may result in a lower dose being required, may reduce side-effects associated with systemic drug delivery, and thus may increase the therapeutic ratio. The drug-ligand conjugate may alternatively be taken up into the cell where it may exhibit its action, either in its conjugate form, or as free drug if a linker labile to intracellular conditions is incorporated into the conjugate. For instance, the linker may be labile to the acid conditions present in. lysosomal compartments. The directing ligand may be a small molecule, peptide, binding protein, antibody, or antibody fragment, identified by one of a number of combinatorial binding screens, for example.

A few examples in the prior art provide for bifunctional drug molecules that combine both elements of a combination therapy regime covalently linked in the same molecule (see for example U.S. Pat. No. 5,780,653).

Linker molecules are commercially available with three homofunctional groups arrayed around a central core (e.g. TMEA—Pierce Chemical Co—Rockford, Ill.) that theoretically allow for such an approach. In practice, however, it is difficult to direct the absolute order of addition of the three components, and complex and lengthy purification steps may be required between the addition of each ligand or pharmacophore.

Another example of the same general principle is the starburst dendrimer system whereby successive rounds of addition to a central homopolyfunctional core results in a large number of reactive groups on the surface of a spherical macromolecule that is available for ligand/drug attachment (see EP0271180). Again, it is difficult to specifically control the ratios of ligand/drug loading with so many possible sites of attachment. In addition, the resultant derivatised dendrimer is extremely large and difficult to quantify exactly.

EP6832096 and Dawson, P. E. et al. (1994; Science 266: 776-799) disclose the ligation of two peptidic compounds to form proteins by a process known as native chemical ligation. The basic requirements for this coupling are the presence on participant A of a thioester moiety such as a benzyl or ethyl thioester, and the presence on participant B of an N-terminal cysteine or functional equivalent containing the minimal grouping of a 1-amino-2 thiol. The coupling reaction commences when the thio-nucleophile initially attacks the thioester in a reversible fashion causing the thioalkyl group to leave. The thioester so formed then undergoes an intramolecular S→N acyl shift to irreversibly produce the ligated amide product A-B with a native cysteine (ie. as found naturally) with a free thiol group at the junction (FIG. 1).

This process can be repeated if it is possible to unmask a further N-terminal cysteine at the N-terminal of A, in the product A-B, by enzymatic means or by classical protecting group chemistry. A further native chemical ligation coupling with a thioester-containing moiety E would therefore yield the product E-A-B. These methods have been used to generate a variety of proteins.

It is also possible to produce cyclic peptides/proteins by incorporating both the thioester and amino terminal cysteine moieties within the same molecule.

It is possible to perform the ligations described above without removing all of the reaction participants from a solid phase.

There is also a prior art method for incorporating a thioester moiety into a participant where one does not exist. This method relies on the reaction of a primary amine with thiolane-2,5-dione and subsequent alkylation of the thioacid formed with an appropriate alkyl bromide. This method does require a single primary amine to be present otherwise multiple thioesters may be coupled.

After a native chemical ligation (NCL) reaction has taken place, there exists at the junction site between A and B (FIG. 1), a nascent cysteine sidechain (ie. a newly formed cysteine sidechain). If neither participant in the NCL reaction contained a free thiol, the free thiol so formed will be the only free thiol in the molecule. No teachings in the prior art recognise the potential for using the free thiol described to react further with a thiol-reactive entity.

There are reports in which considerable effort has been made to remove the cysteine free thiol subsequent to native chemical ligation (WO 98/28434; Yan, L. Z. et al., Journal of the American Chemical Society 123/4: 526-533; Botti, P. et al., 2001, Tetrahedron Letters 42: 1831-1833; Low, D. W. et al., 2001, Proceedings of the National Academy of Sciences of the USA 98[12]:6554-6559).

### DETAIL OF THE INVENTION

In the present invention we show that the free thiol formed by native chemical ligation at the junction of A and B described earlier can in fact be derivatised with suitable reagents to form a branching point. A participant C may form a covalently-linked ‘trimeric’ entity comprising A, B, and C (for example: FIG. 2).

The invention relates to the use of such assemblies in combinatorial ligand discovery. The invention teaches methods of assembling, for example, homo- and heterotrimers and tetramers by native chemical ligation/orthogonal grafting techniques. The invention is exemplified by a number of entities with therapeutic potential, where three or more functionalities are required to be arrayed in a predictable spatial fashion with defined molecular architecture at their junctions. Also described are a number of new classes of molecule that facilitate the assembly of molecular entities that would otherwise be unable to participate.

According to the present invention there is provided a process for forming a compound having the formula (I): 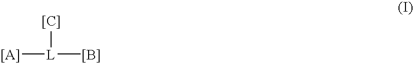
from chemical entities native [A], native [B] and native [C], where native [A] has a thioester group, native [B] has a 1-amino-2-thiol group with an unoxidised sulfhydryl side chain, and native [C] has a thiol reactive function (TRF) group,
wherein chemical entities [A], [B] and [C] are covalently linked by linker group L,
comprising the steps of:
(i) admixing native [A] and native [B] in a reaction solution;
(ii) condensing the unoxidised sulfydryl side chain of native [B] with the thioester group of native [A] for producing a first intermediate compound wherein [A] and [B] are linked with a 5-aminothioester bond;
(iii) rearranging the β-aminothioester bond for producing a second intermediate compound wherein [A] and [B] are linked with an amide bond having attached thereto a free thiol group;
(iv) admixing native [C] with the second intermediate compound in a reaction solution; and
(v) reacting the thiol reactive function (TRF) group of native [C] with the free thiol group of the second intermediate compound for producing a compound having the formula (I).

The terms “native A”, “native B” and “native C” refer to the participant chemical entities in their form before reaction with each other. The “native” entities may thus be regarded as the pre-teaction entities.

Also provided is a process for the synthesis of a compound having the formula (I): 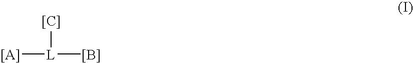
from chemical entities native [A], native [B] and native [C], where native [A] has a thioester group, native [B] has a 1-amino-2-thiol group with an unoxidised sulfhydryl side chain, and native [C] has a thiol reactive function (TRF) group,
wherein chemical entities [A], [B] and [C] are covalently linked by linker group L,
comprising the steps of:
(i) reacting the thioester group of native [A] to the 1-amino-2-thiol group of native [B] by native chemical ligation to form an intermediate compound having the formula (II):
   [A]-L′[B] (II)
   where intermediate linker group L′ comprises an amide bond having attached thereto a free thiol;
and
(ii) reacting the thiol reactive function (TRF) group of native [C] to the free thiol of the intermediate compound having the formula (II) to form the compound having the formula (I).

Yet further provided is a process for the synthesis of a compound having the formula (I): 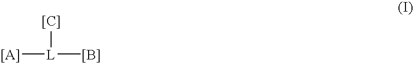
from chemical entities native [A], native [B] and native [C], where native [A] comprises a functional group having a chemical structure, excluding [A], shown in the formula (III): 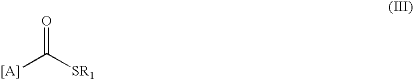
where R₁ is H or a sidechain,
native [B] comprises a functional group having a chemical structure, excluding [B], shown in the formula (IV): 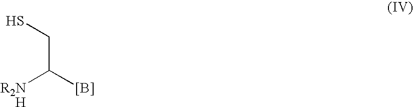
   where R₂ is H or a sidechain,
and native [C] comprises a thiol reactive function (TRF) group attached to [C] as shown in formula (V): 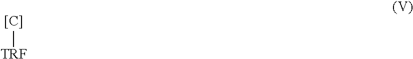
   wherein [A], [B] and [C] are chemical entities covalently linked by a linker group L, and wherein the compound of formula (I) has the chemical structure shown in formula (VI): 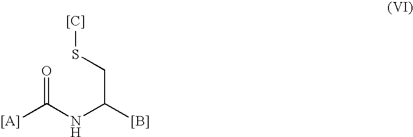
   comprising the steps of:
(i) forming an intermediate compound having the formula (II):
   [A]-L′-[B] (II)
   wherein intermediate linker group L′ comprises a free thiol group, and wherein the intermediate compound of formula (II) has the chemical structure shown in formula (VII): 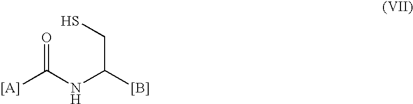
   by reacting the functional group shown in formula (III) of native [A] to the functional group shown in formula (IV) of native [B] using native chemical ligation;
and
(ii) producing the compound having the formula (I) by reacting the thiol reactive function (TRF) group of native [C] to the free thiol of the intermediate linker group L′.

In the above-mentioned process of the invention, native [C] has a thiol reactive function (TRF) group for reacting with the thiol at the junction site between [A] and [B]. An alternative is to provide a thiol reactive group (TRF) linker or spacer linker which reacts with the thiol at the junction site between [A] and [B], then reacting the resultant molecule with native [C]. Thus further provided according to the present invention is a process for forming a compound having the formula (I): 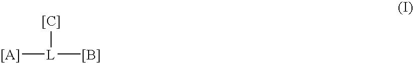
from chemical entities native [A], native [B] and native [C], where native [A] has a thioester group, native [B] has a 1-amino-2-thiol group with an unoxidised sulfhydryl side chain, and native [C] has a thiol group,
wherein chemical entities [A], [B] and [C] are covalently linked by linker group L,
comprising the steps of:
(i) admixing native [A] and native [B] in a reaction solution;
(ii) condensing the unoxidised sulfydryl side chain of native [B] with the thioester group of native [A] for producing a first intermediate compound wherein [A] and [B] are linked with a aminothioester bond;
(iii) rearranging the D-aminothioester bond for producing a second intermediate compound wherein [A] and [B] are linked with an amide bond having attached thereto a free thiol group;
(iv) admixing the second intermediate compound with a thiol reactive function (TRF) group linker having at least two thiol reactive function (TRF) groups in a reaction solution;
(v) allowing the thiol reactive function (TRF) group linker to react with the free thiol group of the second intermediate compound to form a third intermediate compound with a thiol reactive function (TRF) group;
(vi) admixing native [C] with the third intermediate compound in a reaction solution; and
(vii) reacting the thiol group of native [C] with the thiol reactive function (TRF) group of the third intermediate compound for producing a compound having the formula (I).

Also provided is a process for the synthesis of a compound having the formula (I) 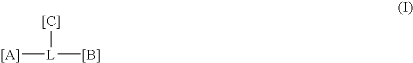
from chemical entities native [A], native [B] and native [C], where native [A] has a thioester group, native [B] has a 1-amino-2-thiol group with an unoxidised sulfhydryl side chain, and native [C] has a thiol,
wherein chemical entities [A], [B] and [C] are covalently linked by linker group L,
comprising the steps of:
(i) reacting the thioester group of native [A] to the 1-amino-2-thiol group of native [B] by native chemical ligation to form a first intermediate compound having the formula (II):
   [A]-L′-[B] (II)
   where intermediate linker group L′ comprises an amide bond having attached thereto a free thiol group;
(ii) reacting a spacer linker having the formula (VIII):
   TRF-Spacer-TRF (VIII)
   wherein TRF is a thiol reactive function (TRF) group and the Spacer is a linking group,
with the free thiol of intermediate linker group L′ to form a second intermediate compound having the formula (IX): 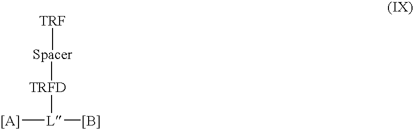
   where TRFD is a thiol reactive function derivative attached to second intermediate linker group L″,
and
(iii) reacting the thiol of native [C] to the thiol reactive function (TRF) group of the second intermediate compound having the formula (IX) to form the compound having the formula (I).

Further provided is a process for the synthesis of a compound having the formula (I): 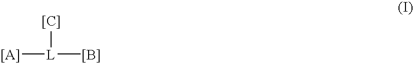
from chemical entities native [A], native [B] and native [C], where native [A] comprises a functional group having a chemical structure, excluding [A], shown in the formula (III): 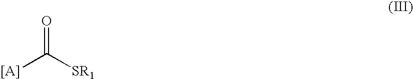
where R₁ is H or a sidechain,
native [B] comprises a functional group having a chemical structure, excluding [B], shown in the formula (IV): 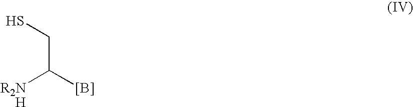
where R₂ is H or a sidechain,
and native [C] comprises a thiol attached to [C] as shown in formula (X): 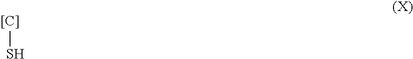
wherein [A], [B] and [C] are chemical entities covalently linked by a linker group L, and wherein the compound of formula (I) has the chemical structure shown in formula (XI): 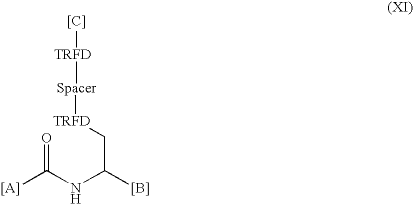
where TRFD is a thiol reactive function derivative and the Spacer is a linking group,
comprising the steps of:
(i) forming an intermediate compound having the formula (II):
   [A]-L′-[B] (II)
   wherein intermediate linker group L′ comprises a free thiol group, and wherein the intermediate compound of formula (II) has the chemical structure shown in formula (VII): 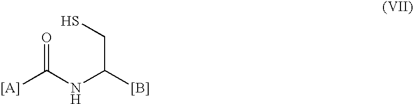
   by reacting the functional group shown in formula (III) of native [A] to the terminal functional group shown in formula (IV) of native [B] using native chemical ligation;
(ii) reacting a spacer linker having the formula (VIII):
   TRF-Spacer-TRF (VIII)
   where TRF is a thiol reactive function (TRF) group and the Spacer is a linking group,
with the free thiol group of intermediate linker group L′ to form a second intermediate compound having the formula (XII): 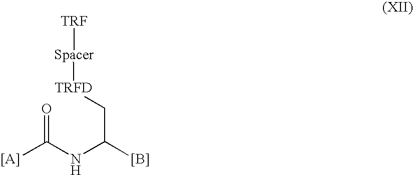
and
(iii) producing the compound having the formula (I) by reacting the thiol of native [C] to the thiol reactive function (TRF) group of the second intermediate compound having the formula (XII).

Alternatively, there is provided a process for forming a compound having the formula (I): 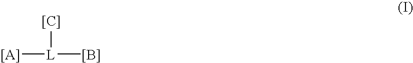
from chemical entities native [A], native [B] and native [C], where native [A] has a thioester group, native [B] has a 1-amino-2-thiol group with an unoxidised sulfhydryl side chain, and native [C] has a thiol reactive function (TRF) group,
wherein chemical entities [A], [B] and [C] are covalently linked by linker group L,
comprising the steps of:
(i) admixing native [A] and native [B] in a reaction solution;
(ii) condensing the unoxidised sulfydryl side chain of native [B] with the thioester group of native [A] for producing a first intermediate compound wherein [A] and [B] are linked with a β-aminothioester bond;
(iii) rearranging the β-aminothioester bond for producing a second intermediate compound wherein [A] and [B] are linked with an amide bond having attached thereto a free thiol group;
(iv) admixing the second intermediate compound with a thiol reactive function (TRF) group linker having a thiol reactive function (TRF) group and a free thiol in a reaction solution;
(v) allowing the thiol reactive function (TRF) group of the thiol reactive function (TRF) group linker to react with the free thiol group of the second intermediate compound to form a third intermediate compound with a free thiol;
(vi) admixing native [C] with the third intermediate compound in a reaction solution; and
(vii) reacting the thiol reactive function (TRF) group of native [C] with the free thiol of the third intermediate compound for producing a compound having the formula (I).

The thiol reactive function (TRF) group linker may comprise, or the Spacer may be, a polyalkyloxy, alkyl, aryl, arylalkyl or peptidyl group.

Each of R₁ and R₂ shown in formula (III) and formula (IV) may be a substituted or unsubstituted aryl group or a substituted or unsubstituted alkyl group.

The functional group of native [B] may be a 1-amino-2-thiol group having an unoxidised sulfhydryl side chain.

The unoxidised sulfhydryl side chain of the 1-amino-2-thiol group of native [B] may be the only unoxidised sulfhydryl side chain present in native [B] . Alternatively, native [B] may comprise additional free thiols which are removed by mutation such that the unoxidised sulfhydryl side chain of the 1-amino-2-thiol group of native [B] becomes the only unoxidised sulfhydryl side chain present.

Native [B] may have a terminal cysteine moiety comprising the terminal 1-amino-2-thiol group.

The thiol reactive function (TRF) group may be a thiol, an alkyl halide, a 2-pyridyl disulfide, a 4-pyridyl disulfide or a Michael acceptor (eg. a vinyl sulfone or a maleimide).

An example of a suitable thiol reactive function (TRF) is an alkyl halide and the reaction is an SN² displacement. The reaction product is a thioether. Incorporation of alkyl halides into various substrates is a straightforward process well known to those skilled in the art.

Another example is an activated disulfide such as a 2- or 4-pyridyl disulfide and the reaction is a disulfide exchange. The reaction product is a disulfide. Pyridyl disulfides may be incorporated synthetically into many substrates. Additionally, a range of reagents is available to install pyridyl disulfides on the surface of proteins and other molecules.

A further example is a Michael acceptor such as a vinyl sulfone or a maleimide and the reaction is a Michael-type addition. The reaction product is a thioether. Maleimides may be incorporated synthetically into many substrates. Additionally, a range of reagents is available to install maleimides on the surface of proteins and other molecules.

Examples of the three resultant reaction products are shown (FIG. 3). Other chemical functions are able to react chemically with free thiols and are well known to those skilled in the art (for example, BMOE).

The thioester group or functional group of native [A] may be a benzyl, ethyl or 2-aminoethyl group.

Native [A] and/or native [B] and/or native [C] may comprise any one of the following entities or derivatives thereof: pharmacophore, ligand, small molecule, purification handle/immunochemical tag (eg biotin, a dinitrophenyl compound), fluorescent moiety (eg. Alexafluor dye, Texas Red dye), solubilising agent, chelating ligand, chelating ligand plus radioimaging agent, therapeutic protein, antibody or fragment thereof, peptide, peptidomimetic or macroscopic particle (eg. a bead).

Prior art (for example U.S. Pat. No. 5,789,653) does not suggest or teach a covalently linked combination therapeutic covalently linked to a directing ligand or antibody or antibody fragment.

Native [A] and/or native [B] and/or native [C] may comprise an entity from a combinatorial array of chemicals. The combinatorial array may be a peptide library.

Also provided according to the present invention is compound obtainable or obtained by the above-mentioned processes.

Further provided is a compound having the general formula (I): 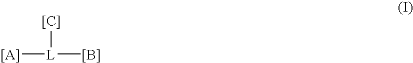
wherein [A], [B] and [C] are derived from chemical entities native [A], native [B] and native [C], respectively, and are covalently linked by linker group L, the compound of formula (I) having a chemical structure shown in formula (VI): 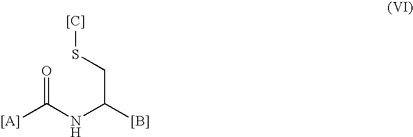
or formula (XI): 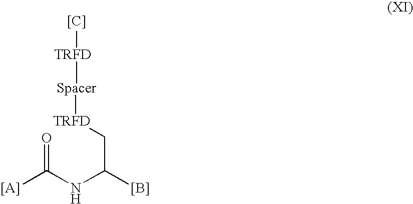
where TRFD is a thiol reactive function derivative and the Spacer is a linking group,
and wherein each of [C], [B] and [C] comprises any one of the following entities or derivatives thereof: pharmacophore, ligand, small molecule, purification handle/immunochemical tag (eg biotin, a dinitrophenyl compound), fluorescent moiety (eg. Alexafluor dye, Texas Red dye), solubilising agent, chelating ligand, chelating ligand plus radioimaging agent, therapeutic protein, antibody or fragment thereof, peptide, peptidomimetic or macroscopic particle (eg. a bead).

Yet further provided is the use of the compound according to the present invention in a binding screen for detecting binding to a target entity. The binding screen may select compounds which have increased or decreased binding to a target entity.

Also provided is the use of a compound formed by native chemical ligation in a process for the production of the compound of the invention.

In one embodiment, native [A] and/or native [B] and/or native [C] may comprise a myristoyl/electrostatic switch peptide (MSWP) or derivative thereof, for example MSWP2278 (SEQ. ID NO: 2).

In another embodiment, native [A] comprises EGFP, native [B] comprises MSWP2278 (SEQ. ID NO: 2) and native [C] comprises Texas Red Dye.

In yet another embodiment, native [A] comprises EGFP, native [B] comprises MSWP2278 (SEQ. ID NO: 2), and native [C] comprises an antibiotic (for example vancomycin).

In an alternative embodiment, native [A] comprises Hirulog anticoagulant peptide, native [B] comprises MSWP2278 (SEQ. ID NO: 2), and native [C] comprises SCR1-3.

WO 98/02454 relates to the derivatisation of soluble complement regulators by disulfide exchange reaction with pyridyl disulfide activated myristoyl/electrostatic switch peptides (MSWPs). These derivatives demonstrate improved complement inhibitory activity owing to their membrane localisation by the MSWP. The myristoyl group was designed to be minimally membrane insertive, while the lysine rich peptidic portion of MSWP-1 (Seq. ID No 1) was designed to interact with the negatively charged phospholipid headgroups present on the lipid bilayer. The concept is exemplified in WO 98/02454 by SCR1-3 of LHR-A of CR1, altered by a single C-terminal cysteine residue. After treatment with an appropriate reducing agent to ensure the presence of a free thiol on the terminal cysteine sidechain, the protein is reacted with MSWP-1 (N-(Myristoyl)-Gly-Ser-Ser-Lys-Ser-Pro-Ser-Lys-Lys-Lys-Lys-Lys-Lys-Pro-Gly-Asp-(S-2-Thiopyridyl) CyS-NH₂) to yield a membrane binding derivative of SCR1-3 (FIG. 4). The resultant conjugate protein showed anti-haemolytic activity orders of magnitude greater than that displayed by the underivatised protein alone.

This chemistry relies on there existing within the protein participant of the reaction a single free thiol resulting in derivatisation at a single point only. There are numerous other cysteines in SCR1-3, but all save the C-terminal cysteine are participants in intramolecular disulfide bridges. Some proteins, however, may have free thiols other than that specific free thiol on which derivatisation is desired to occur. In instances such as this, it may be possible to mutate using molecular biological techniques these cysteine residues to other amino acids, while retaining the desired biological function. In other cases this mutation process may result in the loss of the desired biological function.

WO 98/02454 describes polypeptide derivatives in terms of a combinatorial array of membrane binding elements corresponding to the general structure:
[P]-{L-[W]}ₙ-X 1
in which:
P is the soluble polypeptide,
each L is independently a flexible linker group,
each W is independently a peptidic membrane binding element,
n is an integer of 1 or more, and
X is a peptidic or non-peptidic membrane-binding entity which may be covalently linked to any W.

In the context of the current invention where such an assembly is constructed by native chemical ligation, and where n=1, structure 1 can be rewritten whereby entity A represents [P], entity B represents [W] and X, and thus L is comprised of the nascent cysteine residue at the junction between A and B. We will for simplicity continue our discussion of the scope of the invention using A, B, and C, but with an additional linker function L which is comprised of the nascent cysteine residue at the junction between A and B and any further atoms that may make up a flexible linker to moiety C.

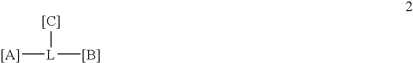

Where [A] is a protein, one method of formation of the [A]-L-[B] portion of the invention is provided by the IMPACT™ system (New England Biolabs). Conjugates are afforded by the native chemical ligation of peptides containing an N-terminal cysteine, to proteins expressed in frame with a controllable intervening peptide sequence (CIVPS) and an affinity purification handle. The desired protein is cleaved from its affinity purification matrix by elution with a free thiol such as MESNA to yield a C-terminal thioester. The MESNA thioester is displaced by the thiol of the N-terminal cysteine before an S→N acyl shift installs the peptide bond in an irreversible fashion. Alternatively, the affinity matrix is incubated with the free peptide, displacing the desired protein from the matrix and ligating in a single step (FIG. 5).

The most favoured MSWP in WO 98/02454 comprised an N-terminal myristoyl function, a C-terminal 2-thiopyridylated cysteine primary amide, and a series of intervening residues including the hexalysine region (FIG. 4). The ligation described above required an N-terminal cysteine not present in MSWP-l. MSWP-l was modified for this purpose to include an N-terminal lysine primary amide modified at its ε-amino function to incorporate a surrogate N-terminal cysteine. This participates in the native chemical ligation while retaining the overall directionality of the so formed tail. This peptide is described as MSWP2278 (Seq. ID No 2). The principle demonstrated by MSWP2278 whereby a peptide may participate in a native chemical ligation via the intermediacy of a 1-amino-2-thiol engineered onto any sidechain using standard solid phase techniques is contained within the scope of the invention.

In the specific case where [C]=[A]-L-[B] it is possible to form a symmetrical homodimer. The alkyl halide, activated disulfide, and maleimide methods described above are also applicable in this case to yield symmetrical thioethers, symmetrical disulfides, and symmetrical maleimides respectively. However a further method also applies. The invention allows for the spontaneous air oxidation of two identical [A]-L-[B] functions to the homodimer in aqueous solution. 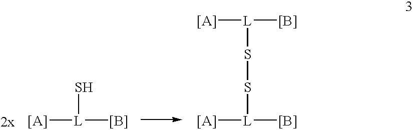
Many other linking chemistry are possible. Some are explained in WO 98/02454 and are incorporated herein by reference.

Clearly if one or more of the following:
A≠A′
B≠B′
is true, then the product of such a coupling reaction will be pseudoheterotetra- or heterotetrameric i.e.

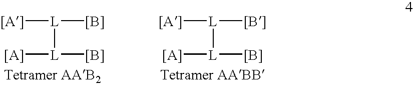

In both of these cases, a simple air oxidation may result in mixtures of products formed, therefore the activated disulfide and Michael type addition methods are preferred. For instance, in the case of the air oxidation of [A]-L-[B]+[A′]-L-[B], the following three products would be observed in the reaction mixture:

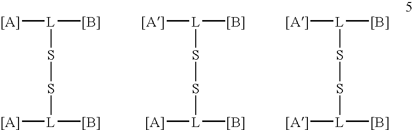

The incorporation of C orthogonally to the existing linear molecular backbone is a fundamental utility described within the invention. The entity C is, as a result of the inventive step, not constrained within the confines of a linear sequence but is attached to a point between the entities A and B.

In the case where one of A, B, or C is a probe such as biotin, or a fluorophore such as an Alexafluor dye, an immunochemical marker such as a dinitrophenyl compound, or a radiochemical label, the trimers or tetramers described above gain utility in that their fate is able to be monitored. These monitoring techniques include but are not limited to an immunohistochemical screen, a fluorescence localisation assay, or radiochemical screen. The scale of monitoring may vary from a whole body screen, to intra-organ differential localisation, to the identification of sub-cellular fate.

An example is:
A=EGFP
B=MSWP2278
C=Texas Red Dye

This heterotrimer (FIG. 6) contains a green fluorescent protein directed towards cell membranes by the myristoyl electrostatic switch peptide. A Texas Red dye was incorporated as [C] using the appropriate maleimide reagent (Molecular Probes—Eugene, Oreg.). This heterotrimer under normal neutral pH conditions in aqueous media fluoresces in both the red and green channels. When incubated with cells, the trimer localises to the cell membrane, where both types of fluorescence may be observed. Some of the heterotrimer passes through the cell membrane and localises to the lysosomal compartments. The low pH found in these compartments abrogates the green fluorescence associated with EGFP, leaving only the Texas Red to fluoresce. This molecule, therefore, can be used as a lysosomal tracking device. Many other applications such as this will become apparent to those skilled in the art through the teachings of this invention.

A further example is:
A=EGFP
B=MSWP2278
C=An antibiotic

The antibiotic in can be vancomycin for example (FIG. 7).

A further example is:
A=Hirulog anticoagulant peptide
B=MSWP2278
C=SCR1-3

This heterotrimer contains all the elements of a ligand directed combination therapy as described earlier. The MSWP2278 peptide serves to localise the remainder of the molecule on the surface of mammalian cells. On these surfaces, the antiocoagulant properties and the complement inhibitory activities of both of the components A and C respectively are conserved. The synthesis was accomplished by the native chemical ligation of MSWP2278 with a hirulog type peptide thioester ((D)-PheProArgProGlyGlyGlyGlyAspGlyAspPheGluGluIleProGluGluTyrLeuGlySerGlySerGlySerGlySerGlySerGlySerGly-SEtCONH₂) (Seq ID No 3). The product formed was reacted with a bismaleimide, and then with SCR1-3. The single exposed C-terminal cysteine reacts solely with the maleimide to provide the heterotrimer (FIG. 8). The complement inhibition activity observed for SCR1-3 linked to MSWP1 was almost completely retained, in addition to an anticoagulant activity associated with a membrane directed hirulog type peptide. The combination of a membrane-directed complement inhibitor and an anti-coagulant in a single therapeutic agent may be useful for applications such as transplantation.

In a further example, C can be incorporated as described above not as a single chemical entity but as a combinatorial array of n chemical entities where C=C(1→n).

Alternatively, this combinatorial array of n chemicals was comprised of a peptide library.

Further, each of n chemical entities was comprised of a different peptide sequence, and was linked to L by disulfide exchange chemistry. This principle permitted the use of the combinatorial array of C(1→n) in a binding screen where the binding energy gained from C was additive and augments that gained from B. The process of identifying binding ligands identifies C sequences capable of increasing the overall binding to the cell type of choice, and thus confers selectivity over other cell types 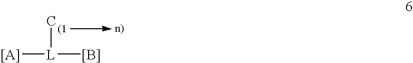
i.e. for library member λ:
ΔG(λ)=Σ(ΔG_{A}+ΔG_{B}+ΔG_{C})

And in the case where B is a MSWP, ΔG_{B}=ΔG_{W}+ΔG_{X}.

There are several possibilities to enable the reduction to practice of such a binding screen. One method involved a one-pot preparation of a multiplicity of library members which were incubated with a tissue for which selective targeting was desired. Each member of this library shall have incorporated into C an affinity handle such as biotin, and shall have a disulfide linkage between C and L. After a washing process to remove library members that either do not bind or bind weakly, the sequence identity of C ligands that bind can be determined by reduction of the disulfide, extraction of C by supported avidin, and identification of C by mass spectrometry.

A different method involves the combinatorial assembly of heterotrimers where the members of the library C(1→n) are used individually in the fashion described above to furnish a library of discrete heterotrimers in a high throughput fashion. The members of this protein library can then be individually screened for binding to a particular cell type in, for instance, a 96-well format. The extent of binding can be detected immunohistochemically (ELISA) or by direct fluorescence in the case where A is fluorescent such as GFP, or where A or B or C or any combination of the three is a fluorescently labeled entity. Both of these methods are exemplified (vide infra)

Any of the components brought together with this enabling technology may be selected from the group consisting of: a pharmacophore, a ligand, a small molecule, a purification handle/immunochemical tag (eg biotin), a fluorescent moiety, a solubilising agent, a chelating ligand, a chelating ligand plus radioimaging agent, a therapeutic protein, an antibody or fragment thereof, a peptide or a peptidomemtic.

This list is not exhaustive. Other moieties may be linked in the fashion to be described and will be apparent to those skilled in the art.

### DRAWINGS

FIG. 1: Native Chemical Ligation schematic.

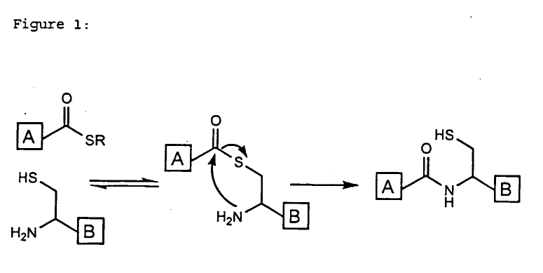

FIG. 2: Formation of a heterotrimer by reaction of a NCL product with a thiol-reactive component.

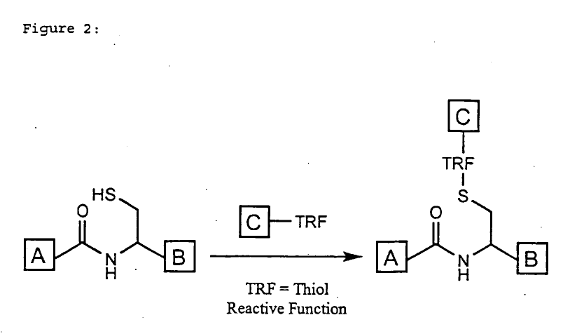

FIG. 3: Examples of heterotrimeric products.

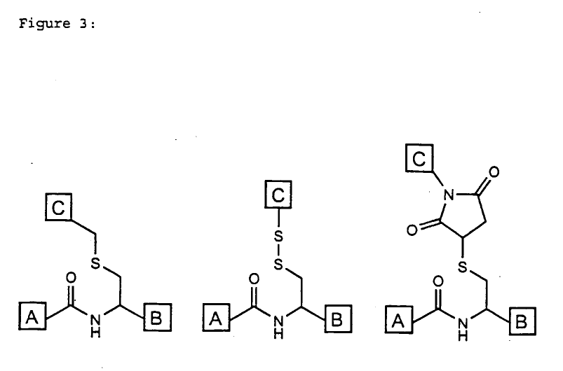

FIG. 4: Preparation of a membrane binding derivative of SC1i-3.

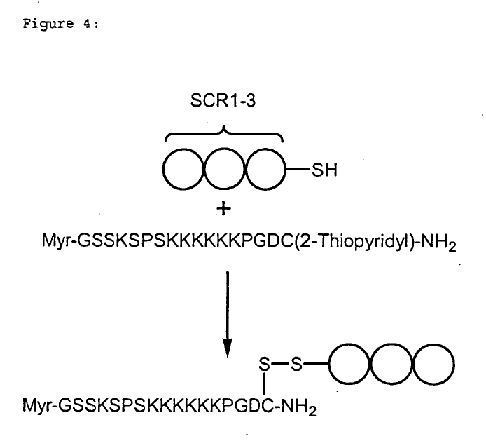

FIG. 5: Ligation of a MSWP to an intein expressed protein.

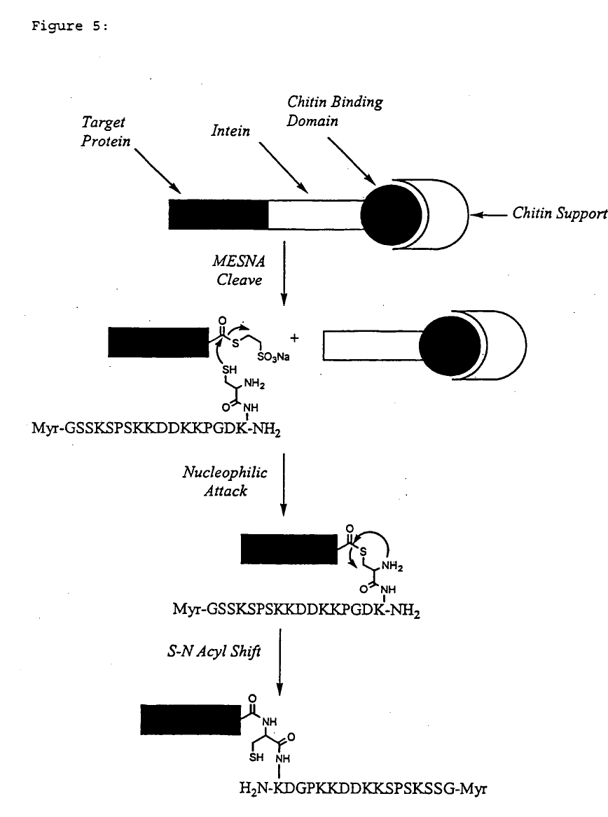

FIG. 6: Two channel fluorescence, pH dependent heterotrimer.

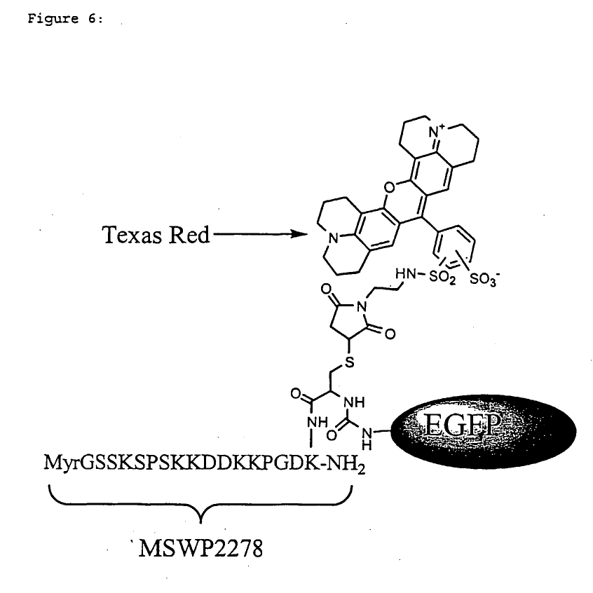

FIG. 7: Membrane-targeted antibiotic/fluorescent protein heterotrimer.

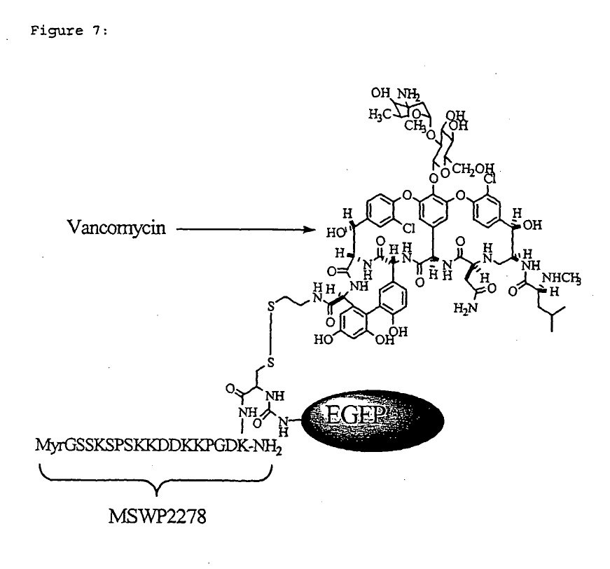

FIG. 8: Membrane-targeted Hirulog anticoagulant/SCR1-3 heterotrimer.

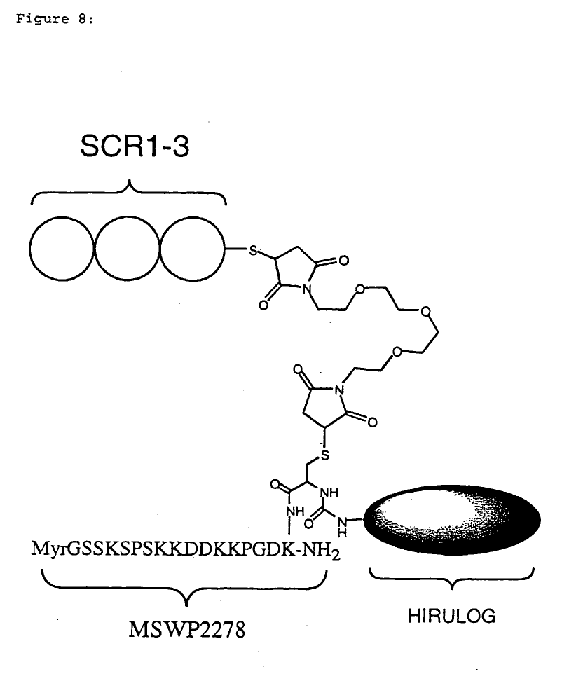

FIG. 9: Binding of untailed and tailed EGFP to COS7 cells. A: Fluorescence micrograph after incubation with EGFP. B: Fluorescence micrograph after incubation with APT2308.

### EXAMPLES

### Example 1

### Synthesis of Disulfide Exchange MSWP1 (SEQ ID NO: 1)

The peptide:
Myristoyl-Gly-Ser-Ser-Lys-Ser-Pro-Ser-Lys-Lys-Lys-Lys-Lys-Lys-Pro-Gly-Asp-Cys-(S-2-thiopyridyl)-NH₂ was synthesised according to the method described in WO 98/02454.

### Example 2

### Synthesis of Native Chemical Ligation/MSWP2278 (SEQ ID NO: 2)

The peptide:
Myristoyl-Gly-Ser-Ser-Lys-Ser-Pro-Ser-Lys-Lys-Asp-Asp-Lys-Lys-Pro-Gly-Asp-(ε-amino-cysteinyl carboxamide)-Lys-NH₂ was prepared by solid phase synthesis using Boc synthesis on MBHA resin (Nova). Coupling reactions were carried out using appropriately protected Boc amino acid monomers (Nova) activated with TBTU aided with HOBT (Alexis Bio. Co.) with ninhydrin monitoring after each extension. The first amino acid was installed as α-amino-Boc, Ε-amino-Fmoc lysine, and the Fmoc protection then removed with 20% piperidine. α-Amino-Fmoc, S-methoxybenzyl cysteine was then coupled to the e-amino group before the remainder of the synthesis was carried out using appropriately protected Boc monomers. Cleavage from the resin and cleavage of the sidechain protecting groups was accomplished with high HF conditions using p-cresol and p-thiocresol as scavengers at 0° C. over 1 h.

The crude peptide was desalted via gel filtration (Sephadex G10, 0.1% TFA in water) before purification by preparative C18 Vydac) high performance liquid chromatography (HPLC) using 0.1% trifluoroacetic acid and 0.1% trifluoroacetic acid/90% acetonitrile as gradient components. After lyophilisation, the peptide was a white amorphous powder, soluble to at least 10 mg/mL in both dimethylsulfoxide and water. Fast atom bombardment spectrometry gave a main peak at 2101 amu corresponding to the molecular ion of the peptide.

### Example 3

### Expression of Recombinant EGFP in the Intein System

Standard molecular biological techniques were used to replace the VP1 gene in vector pET21-VP1Int [Schmidt U., Rudolph, R. & Böhm, G. J. Virol. 74, 1658-1662 (2000)] with the gene for enhanced green fluorescent protein (EGFP), using the vector pEGFP-N1 (Clontech) as template. The resulting vector, pET107-01, expresses the EGFP gene under the control of the T7lac promoter as a C-terminal fusion with the VMA1 gene for the intein from Saccharomyces cerevisiae and the gene for the chitin binding domain from Bacillus circulans. The construction of pET21-VP1Int from vectors pCYB2 and pET21d (New England Biolabs) has been described elsewhere (Schmidt et al., vide supra). Upon transformation into the bacterial strain Hams113, an overnight starter culture of 25 mL LB medium containing 100 μg/mL ampicillin was inoculated with a single colony and, on the next morning, diluted 1:100 into 2 L of LB medium containing 100 μg/mL ampicillin. The culture was fermented at 37° C. until an optical density at 600 nm of 0.5 was achieved, at which point the temperature was lowered to 20° C. and expression of the gene induced by the addition of 1 mM IPTG after 30 mins. Cells were harvested 21 h post induction by low-speed centrifugation and stored in aliquots at −40° C.

### Example 4

### Affinity Purification of EGFP and Tailing with APT542

To lyse cells, an aliquot of bacterial paste corresponding to 333 mL of fermentation broth was resuspended in 40 mL of ice-cold Buffer 160 (20 mM HEPES pH 8.0, 100 mM NaCl, 1 mM EDTA) and homogenised by three passages through an Emulsiflex C5 homogeniser at a pressure of 12 kpsi. The soluble fraction was obtained by centrifugation (20000 g, 30 min, 4° C.) and immediately passed over a column containing 15 mL chitin beads (New England Biolabs). The column was washed with four column volumes of Buffer 100, eight column volumes of Buffer 2000 (20 mM HEPES pH 8.0, 2000 mM NaCl, 1 mM EDTA), four column volumes of Buffer 100, and finally with three column volumes of Cleavage Buffer 1 (Buffer 100 containing 50 mM cysteine). After incubation at 6° C. for 20 h, EGFP was eluted from the column using Buffer 100 as the elution buffer.

The procedure outlined here resulted in an EGFP labelled at the C-terminal with a single additional cysteine residue designated APT2269. The availability of this thiol was tested by derivatisation with the thiol-reactive MSWP1. 500 μL of a 26 μM solution of APT2269 was mixed with 100 μL 10 mM TCEP/20 mM HEPES over 1 h to ensure complete reduction of the terminal cysteine. The mixture was dialysed against 1 L PBS/EDTA (1 mM)/TCEP (1 mM), against 1 L PBS/EDTA (1 mM) and MSWP1 added to a final concentration of 34 SM. This reaction resulted in the derivatisation of APT2269 with between 1 and 3 tails (a process referred to as supertailing).

### Example 5

### Expression of Recombinant EGFP in the Intein System and Tailing with MSWP2278 to Yield APT2308

EGFP was purified as described in Example 4, but Cleavage Buffer 2 (Buffer 100 containing 50 mM MESNA and 50 μM APT2278) was used in the overnight incubation. The cleavage resulted in almost quantitative tailing of EGFP with no evidence of supertailing. Even upon addition of 5 mM APT2278 (equivalent to a 1000-fold molar excess) no further tailing was detected. The reaction product of EGFP with APT2278 was designated APT2308.

### Example 6

### Further Derivatisation of APT2308 with APTS42

To demonstrate the accessibility of the newly introduced thiol in APT2308, we subjected it to further derivatisation with the thiol-reactive compound APT542.

APT2308 was treated with 1 mM TCEP overnight and the buffer exchanged by passing the solution over a Nap-5 column equilibrated with PBS. APT542 was added to a concentration of 13 μM and the reaction mixture analysed by SDS-PAGE after 1 h.

The addition of each tail was confirmed by a 2 kDa shift in molecular weight.

### Example 7

### Binding of APT2308 to COS7 Cells

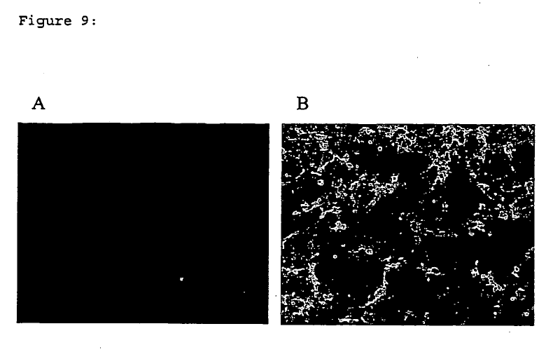

COS7 cells were incubated with either APT2308 or recombinant EGFP at a concentration of 6 μM in Dulbecco's modified eagle medium (Sigma). After 1 h, the cells were visualised by fluorescence microscopy and the images recorded with identical parameters. EGFP demonstrated no binding to COS7 cells (which had been demonstrated with other cell lines for concentrations up to 2.5 mg/mL). APT2308 bound to the cells with very high efficiency (FIG. 9).

### Example 8

### Mutation of EGFP to Remove Two Unpaired Cysteines and Expression in the Intein System—APT2307

pET107-01 was used as a template for mutagenesis with the aim of replacing two cysteine residues in APT 2253 (C49 and C71), with two serine residues. Single base changes (TGC to AGC) were made to each codon at positions 5349 and 5415 of pET107-01.

Mutagenesis primers were designed to extend 15 bases in either direction of each mismatched base. A restriction site was also incorporated into each pair of primers to aid the identification of positive clones. In both cases, this necessitated the introduction of a silent mutation in the coding sequence.

Primers Designed for the Mutation of Cysteine Residue at Position 49 (C49S)

Primer 1200951:
5′ CC CTG AAG TTC ATC AGC ACC ACC GGT AAG CTG CCC

GTG CCC 3′

Primer 1200952:
5′ GGG CAC GGG CAG CTT ACC GGT GGT GCT GAT GAA CTT

CAG GG 3′

In addition to generating a thymine to adenine transition (at position 5349 in pET107-01), these 41mers incorporate an Age I site (ACCGGT) by silent mutation of a glycine codon (G52 in APT 2253), replacing cytosine with thymine (position 5360 in pET107-01).

Primers Designed for the Mutation of Cysteine Residue at Position 71 (C71S).

Primer 1200961:
5′ G ACC TAC GGC GTG CAA AGC TTC AGC CGC TAC C 3′
Primer 1200962:
5′ G GTA GCG GCT GAA GCT TTG CAC GCC GTA GGT C 3′

In addition to generating a thymine to adenine transition (at position 5415 in pET107-01), these 32 mers incorporate a Hind III site (AAGCTT) by silent mutation of a glutamine codon (Q70 in APT 2253), replacing guanine with adenine.

A two step process was used whereby the C49S variant was generated first via site-directed mutagenesis using the QuickChange mutagenising kit (Stratagene), according to the manufacturers instructions. pET107-01 was amplified using primers 1200951 and 1200952, followed by treatment with Dpn I and transformation into XL10 competent cells (Stratagene) to generate pET107-02. pET107-02 was then used as a template for C71S mutagenesis with primers 1200961 and 1200962, using the same process. The resultant plasmid, pET107-04, encodes APT 2307.

MVSKGEELFT GVVPILVELD GDVNGHKFSV SGEGEGDATY GKLTLKFIST TGKLPVPWPT
LVTTLTYGVQ SFSRYPDHMK QHDFFKSAMP EGYVQERTIF FKDDGNYKTR AEVKFEGDTL
VNRIELKGID FKEDGNILGH KLEYNYNSHN VYIMADKQKN GIKVNFKIRH NIEDGSVQLA
DHYQQNTPIG DGPVLLPDNH YLSTQSALSK DPNEKRDHMV LLEFVTAAGI TLGMDELYKP
GCFAKGTNVL MADGSIECIE NIEVGNKVMG KDGRPREVIK LPRGRETMYS VVQKSQHRAH
KSDSSREVPE LLKFTCNATH ELVVRTPRSV RRLSRTIKGV EYFEVITFEM GQKKAPDGRI
VELVKEVSKS YPISEGPERA NELVESYRKA SNKAYFEWTI EARDLSLLGS HVRKATYQTY
APILYENDHF FDYMQKSKFH LTIEGPKVLA YLLGLWIGDG LSDRATFSVD SRDTSLMERV
TEYAEKLNLC AEYKDRKEPQ VAKTVNLYSK VVRGNGIRNN LNTENPLWDA IVGLGFLKDG
VKNIPSFLST DNIGTRETFL AGLIDSDGYV TDENGIKATI KTIHTSVRDG LVSLARSLGL
VVSVNAEPAK VDMNGTKHKI SYAIYMSGGD VLLNVLSKCA GSKKFRPAPA AAFARECRGF
YFELQELKED DYYGITLSDD SDHQFLLGSQ VVVHACGGLT GLNSGLTTNP GVSAWQVNTA
YTAGQLVTYN GKTYKCLQPH TSLAGWEPSN VPAIJWQLQ.

### Example 9

### Tailing of APT2307 with APT2278 to yield APT2360, with APT2335 to Yield APT2361, and with Cysteine to Yield APT2359

Doubly mutated EGFP was tailed using the appropriate peptide or amino acid at 50 μM as described in Example 5. Each reaction mixture was ultrafiltered exhaustively with PBS to remove excess peptide or amino acid.

### Example 10

### Derivatisation of APT2360 with Biotin—APT2396

APT2360 (40 μl of a 10 μM solution in PBS) was mixed with TCEP (2.5 μl of a 1 mM solution in 50 mM HEPES pH 4.5) and left at ambient temperature overnight. EZ-Link PEO-maleimide biotinylation reagent (Pierce, UK). 8 μL of a 400 uM solution in PBS) was added and the mixture left at ambient temperature over 1 h. Freshly made cysteine solution (5 μL of a 10 mM solution in PBS) was added to quench the reaction. Excess reagents were removed by microdialysis. A small gel shift was observed, the new product was immunoreactive with an anti-biotin antibody in a Western Blot, and the expected increase in molecular mass was observed by MALDI mass spec. The reaction proceeded in quantitative yield.

### Example 12

### Derivatisation of APT2360 with an Antibiotic—APT2398

APT2360 was treated with TCEP as described in Example 11 before an activated disulfide derivative of vancomycin (APT2033), at 13.2 μL of a 610 μM solution in PBS, was added. After 2 h at ambient temperature, excess reagents were removed by -microdialysis. A significant gel shift was observed, and the new product was formed quantitatively.

### Example 13

### Derivatisation of APT2361 with a Biotinylated Peptide—APT2404

APT2361 was treated with TCEP as described for APT2360 in Example 11 before a 3-fold molar equivalency of ⁻OOC-MTAAPLRGSXGC(2-S-Py)-NH₂ (X=biotinylated lysine) (APT2400) was added. The mixture was incubated at ambient temperature over 2 h, before excess TCEP and APT2400 were removed by dialysis. An appropriate gel shift was observed by SDS-PAGE corresponding to the addition of a single peptide to the native chemical ligation junction.

### Example 14

### Derivatisation of APT2360 with Texas Red C2 Maleimide—APT2455

APT2360 was prepared as described in Example 9. Upon incubation overnight in the presence of 1 mM TCEP, the sample was dialysed against degassed PBS and Texas Red-maleimide (Molecular Probes) was added to a final concentration of 2 mM (corresponding to an approximate 200-fold excess over APT2360). The mixture was incubated for 2 h at room temperature and then the protein separated from free fluorophore via gel filtration with a PD-10 column. An analysis using SDS-PAGE, UV/VIS spectroscopy and cell binding studies showed that APT2455, the reaction product
absorbed with both fluorophores in neutral to alkaline pH
absorbed only with the Texas Red Fluorophore in acidic pH
bound to cells and caused green and red surface staining as well as predominantly red vesicular staining, due to uptake into acidic lysosomes.

### Example 15

### A Membrane-Targeted Anticoagulant and Complement Inhibitor—APT2434

The production of APT2434 takes place in a three-step synthesis. The first step is the Native Chemical Ligation of APT2383, the hirulog-thioester (including a Glycine-Serine linker segment) to APT2278, yielding APT2384. Typically, both reaction partners are used at mM concentrations and the reaction proceeds overnight at 22 degrees centigrade in the presence of 1 mM TCEP and in 40 mM HEPES buffer, pH 8.5. APT2384 was purified using RP-HPLC and derivatised with a tenfold surplus of BM(PEO)4 in PBS, a bismaleimide with an 18 Å long linker segment (Pierce). The reaction product, APT2433, was purified via RP-HPLC and reacted with APT154 to yield APT2434, the final heterotrimer.

APT2434 was assessed regarding its three proposed functions—membrane localisation, complement inhibition and anticoagulant activity. All three functions were found to be preserved in the final molecule.

1. A process for producing a compound having the formula (I): 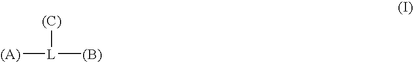
from chemical entities native (A), native (B) and native (C), wherein
(a) native (A) has a thioester group,
(b) native (B) has a 1-amino-2-thiol group with an unoxidized sulfhydryl side chain, and
(c) native (C) has a thiol reactive function (TRF) group,
wherein chemical entities (A), (B) and (C) are covalently linked by linker group l, comprising:
(i) admixing native (A) and native (B) in a reaction solution;
(ii) condensing the unoxidized sulfydryl side chain of native (B) with the thioester group of native (A) for producing a first intermediate compound,
wherein (A) and (B) are linked with a β-aminothioester bond;
(iii) rearranging the β-aminothioester bond for producing a second intermediate compound, wherein (A) and (B) are linked with an amide bond having attached thereto a free thiol group;
(iv) admixing native (C) with the second intermediate compound in a reaction solution; and
(v) reacting the thiol reactive function (TRF) group of native (C) with the free thiol group of the second intermediate compound for producing a compound having the formula (I).

2. A process for the synthesis of a compound having the formula (I): 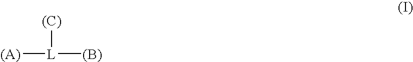
from chemical entities native (A), native (B) and native (C), wherein
(a) native (A) has a thioester group,
(b) native (B) has a 1-amino-2-thiol group with an unoxidized sulfhydryl side chain, and
(c) native (C) has a thiol reactive function (TRF) group,
wherein chemical entities (A), (B) and (C) are covalently linked by linker group L, comprising:
(i) reacting the thioester group of native (A) to the 1-amino-2-thiol group of native (B) by native chemical ligation to produce an intermediate compound having the formula (II):
(A)-L′-(B) (II)
wherein the intermediate linker group L′ comprises an amide bond having attached thereto a free thiol; and
(ii) reacting the thiol reactive function (TRF) group of native (C) with the free thiol of the intermediate compound having the formula (II) to produce the compound having the formula (I).

3. A process for the synthesis of a compound having the formula (I): 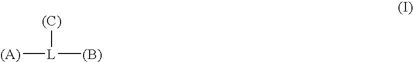
from chemical entities native (A), native (B) and native (C), wherein native (A) comprises a functional group having a chemical structure, excluding (A), shown in the formula (Ill): 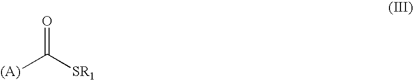
wherein R₁ is H or a side chain,
native (B) comprises a functional group having a chemical structure, excluding (B), shown in the formula (IV): 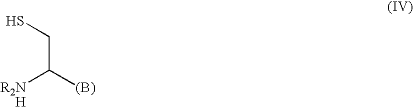
wherein R₂ is H or a side chain,
and native (C) comprises a thiol reactive function (TRF) group attached to (C) as shown in formula (V): 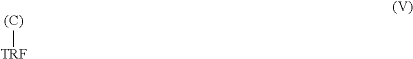
wherein (A), (B) and (C) are chemical entities covalently linked by a linker group L, and wherein the compound of formula (I) has the chemical structure shown in formula (VI): 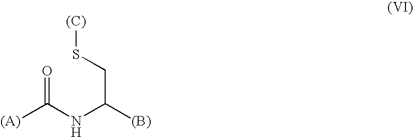
comprising:
(i) forming an intermediate compound having the formula (II):
(A)-L′-(B) (II)
wherein the intermediate linker group L′ comprises a free thiol group, and wherein the intermediate compound of formula (II) has the chemical structure shown in formula (VI): 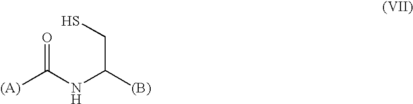
by reacting the functional group shown in formula (Ill) of native (A) with the functional group shown in formula (IV) of native (B) using native chemical ligation; and
(ii) producing the compound having the formula (I) by reacting the thiol reactive function (TRF) group of native (C) with the free thiol of the intermediate linker group L′.

4. A process for producing a compound having the formula (I): 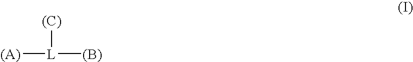
from chemical entities native (A), native (B) and native (C), wherein
(a) native (A) has a thioester group,
(b) native (B) has a 1-amino-2-thiol group with an unoxidized sulfhydryl side chain, and
(c) native (C) has a thiol group,
wherein chemical entities (A), (B) and (C) are covalently linked by linker group L, comprising:
(i) admixing native (A) and native (B) in a reaction solution;
(ii) condensing the unoxidized sulfydryl side chain of native (B) with the thioester group of native (A) for producing a first intermediate compound,
wherein (A) and (B) are linked with a β-aminothioester bond;
(iii) rearranging the β-aminothioester bond for producing a second intermediate compound, wherein (A) and (B) are linked with an amide bond having attached thereto a free thiol group;
(iv) admixing the second intermediate compound with a thiol reactive function (TRF) group linker having at least two thiol reactive function (TRF) groups in a reaction solution;
(v) allowing the thiol reactive function (TRF) group linker to react with the free thiol group of the second intermediate compound to form a third intermediate compound with a thiol reactive function (TRF) group;
(vi) admixing native (C) with the third intermediate compound in a reaction solution; and
(vii) reacting the thiol group of native (C) with the thiol reactive function (TRF) group of the third intermediate compound for producing a compound having the formula (I).

5. A process for the synthesis of a compound having the formula (I): 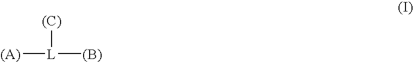
from chemical entities native (A), native (B) and native (C), wherein
(a) native (A) has a thioester group,
(b) native (B) has a 1-amino-2-thiol group with an unoxidized sulfhydryl side chain, and
(c) native (C) has a thiol,
wherein chemical entities (A), (B) and (C) are covalently linked by linker group L, comprising:
(i) reacting the thioester group of native (a) with the 1-amino-2-thiol group of native (B) by native chemical ligation to form a first intermediate compound having the formula (II):
(A)-L′-(B) (II)
wherein intermediate linker group L′ comprises an amide bond having attached thereto a free thiol group;
(ii) reacting a spacer linker having the formula (VIII):
TRF-Spacer-TRF (VIII)
wherein TRF is a thiol reactive function (TRF) group, and the Spacer is a linking group, wherein the free thiol of intermediate linker group L′ forms a second intermediate compound having the formula (IX): 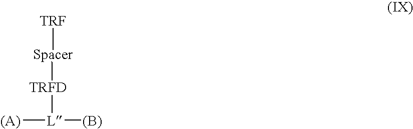
wherein TRFD is a thiol reactive function derivative attached to the second intermediate linker group L″, and
(iii) reacting the thiol of native (C) with the thiol reactive function (TRF) group of the second intermediate compound having the formula (IX) to produce the compound having the formula (I).

6. A process for the synthesis of a compound having the formula (I): 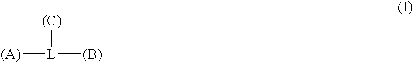
from chemical entities native (A), native (B) and native (C), wherein native (A) comprises a functional group having a chemical structure, excluding (A), shown in the formula (III): 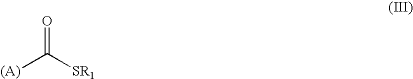
wherein R₁ is H or a side chain,
native (B) comprises a functional group having a chemical structure, excluding (B), shown in the formula (IV): 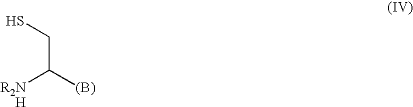
wherein R₂ is H or a side chain,
and native (C) comprises a thiol attached to (C) as shown in formula (X): 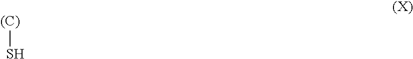
wherein (A), (B) and (C) are chemical entities covalently linked by a linker group L, and wherein the compound of formula (I) has the chemical structure shown in formula (XI): 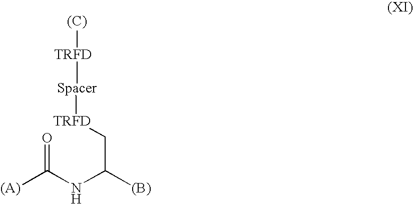
wherein TRFD is a thiol reactive function derivative, and the Spacer is a linking group, comprising:
(i) forming an intermediate compound having the formula (II):
(A)-L′-(B) (II)
wherein the intermediate linker group L′ comprises a free thiol group, and wherein the intermediate compound of formula (II) has the chemical structure shown in formula (VII): 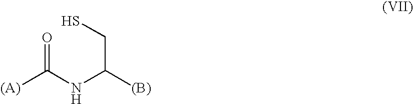
by reacting the functional group shown in formula (III) of native (A) with the terminal functional group shown in formula (IV) of native (B) using native chemical ligation;
(ii) reacting a spacer linker having the formula (VIII):
TRF-Spacer-TRF (VIII)
wherein TRF is a thiol reactive function (TRF) group and the Spacer is a linking group, with the free thiol group of intermediate linker group L′ to form a second intermediate compound having the formula (XII): 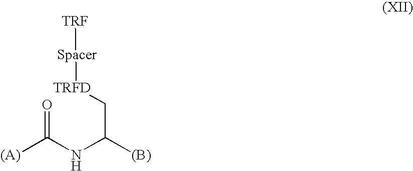
and
(iii) producing the compound having the formula (I) by reacting the thiol of native (C) with the thiol reactive function (TRF) group of the second intermediate compound having the formula (XII).

7. A process for producing a compound having the formula (I): 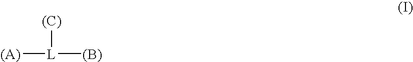
from chemical entities native (A), native (B) and native (C), wherein
(a) native (A) has a thioester group,
(b) native (B) has a 1-amino-2-thiol group with an unoxidized sulfhydryl side chain, and
(c) native (C) has a thiol reactive function (TRF) group,
wherein chemical entities (A), (B) and (C) are covalently linked by linker group L, comprising:
(i) admixing native (A) and native (B) in a reaction solution;
(ii) condensing the unoxidized sulfydryl side chain of native (B) with the thioester group of native (A) for producing a first intermediate compound, wherein (A) and (B) are linked with a β-aminothioester bond;
(iii) rearranging the β-aminothioester bond for producing a second intermediate compound, wherein (A) and (B) are linked with an amide bond having attached thereto a free thiol group;
(iv) admixing the second intermediate compound with a thiol reactive function (TRF) group linker having a thiol reactive function (TRF) group and a free thiol in a reaction solution;
(v) allowing the thiol reactive function (TRF) group of the thiol reactive function (TRF) group linker to react with the free thiol group of the second intermediate compound to produce a third intermediate compound with a free thiol;
(vi) admixing native (C) with the third intermediate compound in a reaction solution; and
(vii) reacting the thiol reactive function (TRF) group of native (C) with the free thiol of the third intermediate compound for producing a compound having the formula (I).

8. The process of claim 7, wherein the thiol reactive function (TRF) group linker comprises, a polyalkyloxy, alkyl, aryl, arylalkyl₁ or peptidyl group.

9. The process of claim 6, wherein each of R₁ and R₂ is a substituted aryl group.

10. The process of claim 9 wherein the functional group of native (B) is a 1-amino-2-thiol group having an unoxidized sulfhydryl side chain.

11. The process of claim 10, wherein the unoxidized sulfhydryl side chain of the 1-amino-2-thiol group of native (B) is the only unoxidized sulfhydryl side chain present in native (B).

12. The process of claim 10, wherein native (B) comprises additional free thiols which are removed by mutation such that the unoxidized sulfhydryl side chain of the 1-amino-2-thiol group of native (B) becomes the only unoxidized sulfhydryl side chain present.

13. The process of claim 12, wherein native (B) has a terminal cysteine moiety comprising the terminal 1 -amino-2-thiol group.

14. The process of claim 13, wherein the thiol reactive function (TRF) group is
(a) a thiol,
(b) an alkyl halide,
(c) a 2-pyridyl disulfide,
(d) a 4-pyridyl disulfidea or
(e) a Michael acceptor.

15. The process of e claim 14, wherein the thioester group of native (A) is a benzyl group.

16. The process of claim 15, wherein native (A) and/or native (B) and native (C) comprise any one of the following entities or derivatives thereof: pharmacophore, ligand, small molecule, purification handle or immunochemical tag, fluorescent moiety, solubilizing agent, chelating ligand, chelating ligand plus radioimaging agent, therapeutic protein, antibody or fragment thereof, peptide, peptidomimetic or macroscopic particle.

17. The process of claim 16, wherein native (A), native (B) and native (C) comprise an entity from a combinatorial array of chemicals.

18. The process claim 17, wherein said combinatorial array is a peptide library.

19. A compound obtainable by the process of claim 18.

20. A compound obtained by the process of claim 18.

21. A compound having the general formula (I): 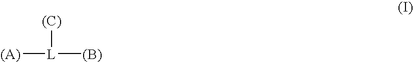
wherein (A), (B) and (C) are derived from chemical entities native (A), native (B), and native (C), respectively, and are covalently linked by linker group L, wherein the compound of formula (I) has the chemical structure shown in formula (VI): 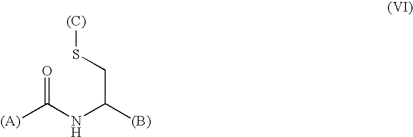
or formula (XI): 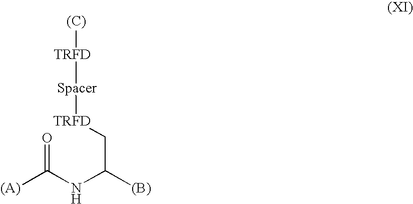
wherein TRFD is a thiol reactive function derivatives and the Spacer is a linking group, and wherein each of (A), (B) and (C) comprises any one of the following entities or derivatives thereof: pharmacophore, ligand, small molecule, purification handle or immunochemical tag fluorescent moiety solubilizing agent, chelating ligand, chelating ligand plus radioimaging agent, therapeutic protein, antibody or fragment thereof, peptide, peptidomimetic or macroscopic particle.

22. A method of using the compound of claim 21 in a binding screen for detecting binding to a target entity.

23. The to method of claim 22, wherein the binding screen selects compounds that have increased binding to a target entity.

24. A method of using a compound formed by native chemical ligation in the process of claim 20.

25. The process, of claim 24, wherein native (C) has
(a) the structure of the second intermediate compound wherein the β-aminothioester bond has been rearranged, and wherein (A) and (B) are linked with an amide bond having attached thereto a free thiol group;
(b) the structure of the intermediate compound of formula (II),
(A)-L′ -(B) (II)
(c) the structure of the third intermediate compound, wherein the thiol reactive function (TRF) group linker is allowed to react with the free thiol group of the second intermediate compound to form a third intermediate compound with a thiol reactive function (TRF) group,
(d) the structure of the second intermediate compound of formula (IX), 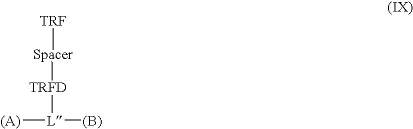
(e) the structure of the second intermediate compound of formula (XII), 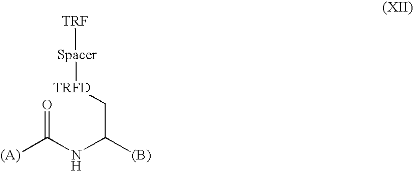
wherein the compound is a symmetrical homodimer.

26. The process, of claim 4, wherein the compound is a tetramer.

27. The process of claim 26, wherein the compound is formed by spontaneous air oxidation between thiol groups or thiol reactive function (TRF) groups.

28. The process, of claim 27, wherein native (A), native (B), and native (C) comprise a myristoy or electrostatic switch peptide (MSWP) or derivative thereof.

29. The process, of claim 28, wherein
(a) native (A) comprises EGFP,
(b) native (B) comprises MSWP2278 (SEQ. ID NO: 2), and
(c) native (C) comprises Texas Red Dye.

30. The process, of claim 28, wherein
(a) native (A) comprises EGFP,
(b) native (B) comprises MSWP2278 (SEQ. ID NO: 2), and
(c) native (B) comprises an antibiotic.

31. The process, of claim 28, wherein
(a) native (A) comprises Hirulog anticoagulant peptide,
(b) native (B) comprises MSWP2278 (SEQ. ID O:2), and
(c) native (B) comprises SCR1-3.

32. The process of claim 7, wherein the spacer comprises a polyalkyloxy, alkyl, aryl, arylalkyl, or peptidyl group.

33. The process of claim 6, wherein each of R₁ and R₂ is an unsubstituted aryl group.

34. The process of claim 6, wherein each of R₁ and R₂ is a substituted alkyl group.

35. The process of claim 6, wherein each of R₁ and R₂ is an unsubstituted alkyl group.

36. The process of claim 3, wherein each of R₁ and R₂ is a substituted aryl group.

37. The process of claim 3, wherein each of R₁ and R₂ is an unsubstituted aryl group.

38. The process of claim 3, wherein each of R₁ and R₂ is a substituted alkyl group.

39. The process of claim 3, wherein each of R₁ and R₂ is an unsubstituted alkyl group.

40. The process of claim 3, wherein the functional group of native (B) is a 1-amino-2-thiol group having an unoxidized sulfhydryl side chain.

41. The process of claim 6, wherein the functional group of native (B) is a 1-amino-2-thiol group having an unoxidized sulfhydryl side chain.

42. The process of claim 1, wherein the unoxidized sulfhydryl side chain of the 1 -amino-2-thiol group of native (B) is the only unoxidized sulfhydryl side chain present in native (B).

43. The process of claim 2, wherein the unoxidized sulfhydryl side chain of the 1-amino-2-thiol group of native (B) is the only unoxidized sulfhydryl side chain present in native (B).

44. The process of claim 4, wherein the unoxidized sulfhydryl side chain of the 1-amino-2-thiol group of native (B) is the only unoxidized sulfhydryl side chain present in native (B).

45. The process of claim 5, wherein the unoxidized sulfhydryl side chain of the 1-amino-2-thiol group of native (B) is the only unoxidized sulfhydryl side chain present in native (B).

46. The process of claim 7, wherein the unoxidized sulfhydryl side chain of the 1 -amino-2-thiol group of native (B) is the only unoxidized sulfhydryl side chain present in native (B)

47. The process of claim 1, wherein native (B) comprises additional free thiols which are removed by mutation such that the unoxidized sulfhydryl side chain of the 1-amino-2-thiol group of native (B) becomes the only unoxidized sulfhydryl side chain present.

48. The process of claim 2, wherein native (B) comprises additional free thiols which are removed by mutation such that the unoxidized sulfhydryl side chain of the 1-amino-2-thiol group of native (B) becomes the only unoxidized sulfhydryl side chain present.

49. The process of claim 4, wherein native (B) comprises additional free thiols which are removed by mutation such that the unoxidized sulfhydryl side chain of the 1-amino-2-thiol group of native (B) becomes the only unoxidized sulfhydryl side chain present.

50. The process of claim 5, wherein native (B) comprises additional free thiols which are removed by mutation such that the unoxidized sulfhydryl side chain of the 1-amino-2-thiol group of native (B) becomes the only unoxidized sulfhydryl side chain present.

51. The process of claim 7, wherein native (B) comprises additional free thiols which are removed by mutation such that the unoxidized sulfhydryl side chain of the 1-amino-2-thiol group of native (B) becomes the only unoxidized sulfhydryl side chain present.

52. The process of claim 1, wherein native (B) has a terminal cysteine moiety comprising the terminal 1-amino-2-thiol group.

53. The process of claim 2, wherein native (B) has a terminal cysteine moiety comprising the terminal 1-amino-2-thiol group.

54. The process of claim 4, wherein native (B) has a terminal cysteine moiety comprising the terminal 1-amino-2-thiol group.

55. The process of claim 5, wherein native (B) has a terminal cysteine moiety comprising the terminal 1-amino-2-thiol group.

56. The process of claim 7, wherein native (B) has a terminal cysteine moiety comprising the terminal 1-amino-2-thiol group.

57. The process of claim 14, wherein the Michael acceptor is a vinyl sulfone.

58. The process of claim 14, wherein the Michael acceptor is a maleimide.

59. The process of claim 14, wherein the thioester group of native (A) is an ethyl group.

60. The process of claim 14, wherein the thioester group of native (A) is a 2-aminoethyl group.

61. The process of claim 14, wherein the functional group of native (A) is a benzyl group.

62. The process of claim 14, wherein the functional group of native (A) is an ethyl group.

63. The process of claim 14, wherein the functional group of native (A) is a 2-aminoethyl group.

64. The process of claim 16, wherein the purification handle or immunochemical tag is biotin.

65. The process of claim 16, wherein the purification handle or immunochemical tag is a dinitrophenyl compound.

66. The process of claim 16, wherein the fluorescent moiety is Alexafluor dye.

67. The process of claim 16, wherein the fluorescent moiety is Texas Red dye.

68. The process of claim 16, wherein the peptidomimetic or macroscopic particle is a bead.

69. The process of claim 21, wherein the purification handle or immunochemical tag is biotin.

70. The process of claim 21, wherein the purification handle or immunochemical tag is a dinitrophenyl compound.

71. The process of claim 21, wherein the fluorescent moiety is Alexafluor dye.

72. The process of claim 21, wherein the fluorescent moiety is Texas Red dye.

73. The process of claim 21, wherein the peptidomimetic or macroscopic particle is a bead.

74. The method of claim 22, wherein the binding screen selects compounds that have decreased binding to a target entity.

75. The compound of claim 24, wherein native (C) has
(a) the structure of the second intermediate compound, wherein the β-aminothioester bond has been rearranged, and wherein (A) and (B) are linked with an amide bond having attached thereto a free thiol group;
(b) the structure of the intermediate compound of formula (II),
(A)-L′-(B) (II)
(c) the structure of the third intermediate compound, wherein the thiol reactive function (TRF) group linker is allowed to react with the free thiol group of the second intermediate compound to form a third intermediate compound with a thiol reactive function (TRF) group,
(d) the structure of the second intermediate compound of formula (IX), 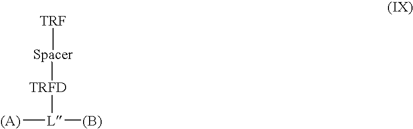
(e) the structure of the second intermediate compound of formula (XII), 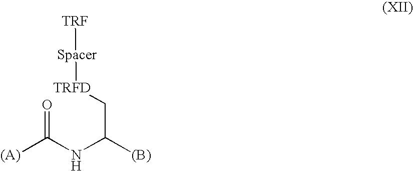
wherein the compound is a symmetrical homodimer.

76. The method of claim 24, wherein native (C) has
(a) the structure of the second intermediate compound, wherein the β-aminothioester bond has been rearranged, and wherein (A) and (B) are linked with an amide bond having attached thereto a free thiol group;
(b) the structure of the intermediate compound of formula (II),
(A)-L′-(B) (II)
(c) the structure of the third intermediate compound, wherein the thiol reactive function (TRF) group linker is allowed to react with the free thiol group of the second intermediate compound to form a third intermediate compound with a thiol reactive function (TRF) group,
(d) the structure of the second intermediate compound of formula (IX), 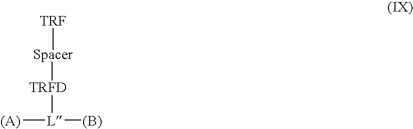
(e) the structure of the second intermediate compound of formula (XII), 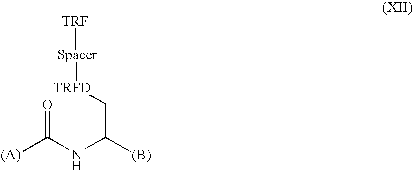
wherein the compound is a symmetrical homodimer.

77. The compound of claim 24, wherein the compound is a tetramer.

78. The method of claim 24, wherein the compound is a tetramer.

79. The process of claim 28, wherein the myristoyl or electrostatic switch peptide (MSWP) or derivative thereof is MSWP2278 (SEQ. ID NO: 2).

80. The compound of claim 27, wherein native (A), native (B), and native (C) comprise a myristoyl or electrostatic switch peptide (MSWP) or derivative thereof.

81. The compound of claim 81, wherein the myristoyl or electrostatic switch peptide (MSWP) or derivative thereof is MSWP2278 (SEQ. ID NO: 2).

82. The method of claim 78, wherein native (A), native (B), and native (C) comprise a myristoyl or electrostatic switch peptide (MSWP) or derivative thereof.

83. The method of claim 82, wherein the myristoyl or electrostatic switch peptide (MSWP) or derivative thereof is MSWP2278 (SEQ. ID NO: 2).

84. The compound of claim 28, wherein
(a) native (A) comprises EGFP,
(b) native (B) comprises MSWP2278 (SEQ. ID NO: 2), and
(c) native (C) comprises Texas Red Dye.

85. The method of claim 83, wherein
(a) native (A) comprises EGFP,
(b) native (B) comprises MSWP2278 (SEQ. ID NO: 2), and
(c) native (C) comprises Texas Red Dye.

86. The process of claim 30, wherein the antibiotic is vancomycin.

87. The compound of claim 28, wherein
(a) native (A) comprises EGFP,
(b) native (B) comprises MSWP2278 (SEQ. ID NO: 2), and
(c) native (C) comprises an antibiotic.

88. The compound of claim 87, wherein the antibiotic is vancomycin.

89. The method of claim 83, wherein
(a) native (A) comprises EGFP,
(b) native (B) comprises MSWP2278 (SEQ. ID NO: 2), and
(c) native (C) comprises an antibiotic.

90. The method of claim 89, wherein the antibiotic is vancomycin.

91. The compound of claim 28, wherein
(a) native (A) comprises Hirulog anticoagulant peptide,
(b) native (B) comprises MSWP2278 (SEQ. ID O: 2), and
(c) native (C) comprises SCR1-3.

92. The method of claim 90, wherein
(a) native (A) comprises Hirulog anticoagulant peptide,
(b) native (B) comprises MSWP2278 (SEQ. ID O: 2), and
(c) native (C) comprises SCR1-3.
